SEQUENCE LISTING (2 sequences)

SEQ ID NO: 1 - protein, 261 aa, Homo sapiens
  MISC_FEATURE: prostate specific antigen
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 2 - protein, 555 aa, Homo sapiens
  MISC_FEATURE: spermine oxidase
MQSCESSGDSADDPLSRGLRRRGQPRVVVIGAGLAGLAAAKALLEQGFTDVTVLEASSHIGGRVQSVKLGHATFELGATWIHGSHGNPIYHLAEANGLLEETTDGERSVGRISLYSKNGVACYLTNHGRRIPKDVVEEFSDLYNEVYNLTQEFFRHDKPVNAESQNSVGVFTREEVRNRIRNDPDDPEATKRLKLAMIQQYLKVESCESSSHSMDEVSLSAFGEWTEIPGAHHIIPSGFMRVVELLAEGIPAHVIQLGKPVRCIHWDQASARPRGPEIEPRGEGDHNHDTGEGGQGGEEPRGGRWDEDEQWSVVVECEDCELIPADHVIVTVSLGVLKRQYTSFFRPGLPTEKVAAIHRLGIGTTDKIFLEFEEPFWGPECNSLQFVWEDEAESHTLTYPPELWYRKICGFDVLYPPERYGHVLSGWICGEEALVMEKCDDEAVAEICTEMLRQFTGNPNIPKPRRILRSAWGSNPYFRGSYSYTQVGSSGADVEKLAKPLPYTESSKTAPMQVLFSGEATHRKYYSTTHGALLSGQREAARLIEMYRDLFQQGT

(19) United States
(12) Patent Application Publication
(10) Pub. No.: US 2021/0270836 A1
(43) Pub. Date: Sep. 2, 2021

(54) METHOD FOR THE IN VITRO DIAGNOSIS OF PROSTATE CANCER BY MEANS OF URINARY BIOMARKERS

(71) Applicant: NIB BIOTEC S.R.L., Torino (IT)

(72) Inventor: Sergio Occhipinti, Torino (IT)

(21) Appl. No.: 17/260,465

(22) PCT Filed: Jul. 17, 2019
(86) PCT No.: PCT/IB2019/056111

§ 371 Date: Jan. 14, 2021

(30) Foreign Application Priority Data
Jul. 17, 2018 (IT) 102018000007264

(51) Int. Cl. G01N 33/574; G01N 33/493; G01N 21/25

(52) U.S. Cl. CPC G01N 33/57434; G01N 2333/90677; G01N 21/251; G01N 33/493

(57) ABSTRACT

The present invention concerns a method for the in vitro/ex vivo diagnosis of a prostate cancer and/or the aggressiveness thereof by means of urinary biomarkers.

### CROSS-REFERENCE TO RELATED APPLICATIONS

This patent application claims priority from Italian patent application no. 102018000007264 filed on Jul. 17, 2018, the entire disclosure of which is incorporated herein by reference.

### TECHNICAL FIELD

The present invention concerns a method for the in vitro/ex vivo diagnosis of prostate cancer and/or the aggressiveness thereof by means of urinary biomarkers.

### STATE OF THE ART

Prostate cancer (PCa) represents the commonest pathology and the second cause of cancer-related death for men worldwide.

Currently the diagnosis of prostate cancer is made only after histological evaluation of the tumour on various prostate tissue samples taken by biopsy. Prostate biopsy is indicated following evaluation of the circulating levels of the prostate specific antigen (PSA) together with digital rectal examination (DRE).

PSA and DRE are preliminary examinations with the object of identifying patients most likely to have developed a prostate tumour. In the presence of abnormalities, the prostate biopsy is performed to confirm or disprove the clinical evidence. However, the limited accuracy of these tests leads to a high number of over-diagnoses and consequently over-treatments.

On average, in fact, for every four men who undergo a prostate biopsy due to suspect PSA levels, one single individual is affected by prostate cancer after bioptic examination.

Furthermore, many of the prostate cancers diagnosed have a very slow course and may not show symptoms for the whole of the patient's life. Since it is impossible to distinguish the over-diagnosed tumours from the others, the majority of patients found positive to the screening are offered surgery which is often accompanied by adverse effects (such as erectile dysfunction, urinary incontinence, infections) with a strong impact on the quality of life.

In addition to patient inconvenience, the economic impact of such a high percentage of unnecessary tests on health system resources should be considered. One of the ways of assessing if good use is being made of resources (good value for money) is to express the clinical benefits in relation to the economic aspects. The two economic studies existing on screening with PSA agree in defining this operation to be non-cost-effective.

In short, it means that on the entire population the costs connected with the process implemented once the PSA test has been performed (due to biopsies, treatments, possible damage) outweigh the benefits, quantified in economic terms.

In the light of the above, it is clear that specific biomarkers for the diagnosis of prostate cancer need to be urgently identified to pinpoint men at risk of developing prostate cancer and predict the natural progression of the tumour.

Some recent studies suggest that male infertility can be a signal of the risk of developing an aggressive form of prostate cancer. In particular it has been seen that patients with a diagnosis of infertility are three times more likely to develop an aggressive prostate tumour than the fertile male population (Walsh T J et al., Cancer. 2010 May 1; 116(9):2140-7).

The prostate is a gland with the main function of producing the seminal liquid containing elements fundamental for the survival, motility and quality of the spermatozoa. It has been seen that chronic inflammatory conditions cause alterations in the physical-chemical characteristics of the liquid part of the ejaculate (variations in viscosity and fluidification, in pH, presence of white globules, modifications in the levels of PSA, PAP, zinc, fructose and citric acid) affecting fertility.

During neoplastic transformation, the cell can undergo profound phenotypic and functional changes. Since the majority of prostate tumours are classified as adenocarcinomas, namely tumours that involve the glandular part, it is plausible that the neoplastic transformation within the prostate can jeopardize the secretory function of the gland, compromising the composition of the prostatic secretion.

From some studies it has emerged that advanced prostate tumours can lose the expression of the PSA (Aihara M et al., J Urol. 1994; 151:1558-1564; Weir E G et al., J Urol. 2000; 163:1739-1742; Augustin H et al., J Cancer Res Clin Oncol. 2003; 129:662-668). More detailed studies using the tissue microarray technology have confirmed that the expression of the PSA in the tissue can be used both as a diagnostic and prognostic parameter for prostate cancer (Erbersdobler A et al., Urology. 2009 November; 74(5):1169-73).

Since the urinary tract is in close contact with the prostate, the factors produced by the prostatic tissue can be transferred and therefore detected in the urine, representing biological markers for the diagnosis and prognosis of prostate cancer.

On the basis of this, measurement of the urinary PSA should provide useful information concerning the physiology and pathological situation of the prostate, since the PSA is the normal product of the epithelial cells that surround the prostatic acini and ducts (Irani J et al., Eur Urol 1996; 29:407-12).

EP2515115 describes use of the urinary PSA as a prostate tumour marker. In EP2515115, however, the absence of prostate massage prior to the collection of urine does not allow a consistent prostatic secretion to be obtained. The mean values identified are around 50 ng/ml for healthy subjects and 5 ng/ml for those affected by tumour.

In a work published in 2007 it is observed that the ratio between urinary PSA and plasmatic PSA is different between patients with prostate tumour, patients with benign hyperplasia of the organ and healthy subjects (Bolduc S et al., Can Urol Assoc J. 2007 November; 1(4):377-81).

Recently it has been seen that the circulating PSA exists in a free form and a form complexed to different molecules of the protease inhibitor family. More frequently, the PSA is complexed to the alpha-1-antichymotripsin (ACT) and it has been observed that patients with prostate tumour have a higher percentage of PSA bound with ACT than healthy donors who show, on the other hand, a higher percentage of free PSA (Christensson A et al., J Urol. 1993; 150:100-5).

Various studies have shown at tissue level a higher expression of ACT as mRNA and protein in prostate tumour compared to benign hyperplasia, justifying the higher quantity of complexed PSA (Zhu L et al., Prostate. 2013 January; 73(2):219-26).

WO0002052 describes use of the PSA, complexed and free, cleaved and non-cleaved, as biomarkers in plasma/serum to distinguish a healthy subject from a subject with prostate cancer.

### DISCLOSURE OF INVENTION

The object of the present invention is to provide a method for the in vitro/ex vivo diagnosis of prostate cancer by means of new biomarkers present in urine, which provides reliable and accurate results rapidly and inexpensively.

According to the present invention, said object is achieved by means of the method according to claims 1 and 4.

According to the present invention a kit for use in the above-mentioned method is also provided.

### BRIEF DESCRIPTION OF THE FIGURES

For a better understanding of the present invention, it is now described also with reference to the attached figures, which illustrate the following:

FIG. 1 shows the graphs of the absolute values of the urinary biomarkers Zinc (Zn), total PSA (utPSA), free PSA (ufPSA), spermine, spermine oxidase (SMOX) in individuals affected by prostate cancer and in healthy individuals;

FIG. 2 shows the graphs of the values of the urinary biomarkers Zn, utPSA, ufPSA, spermine (Spm), spermine oxidase (SMOX) normalized on creatinine in individuals affected by prostate cancer and in healthy individuals;

FIG. 3 shows the ROC (receiver operating characteristic) curve for Zn, spermine (Spm), utPSA, ufPSA, spermine oxidase (SMOX);

FIG. 4a shows the ROC curve for the combinations Zn-Spm, Zn-utPSA, Zn-ufPSA, ufPSA-Spm, utPSA-ufPSA, utPSA-Spm;

FIG. 4b shows the ROC curve for the combinations Zn-Spm-utPSA, Zn-Spm-ufPSA, Zn-utPSA-ufPSA, Spm-utPSA-ufPSA, Spm-utPSA-ufPSA-Zn;

FIG. 4c shows the ROC curve for the combinations Zn-Spm-SMOX, Zn-utPSA-SMOX, Zn-ufPSA-SMOX, ufPSA-Spm-SMOX, utPSA-Spm-SMOX, Zn-utPSA-SMOX-Spm;

FIG. 5 shows the graphs of the absolute values of the urinary biomarkers Zinc (Zn), total PSA (utPSA), free PSA (ufPSA), spermine, spermine oxidase (SMOX) in individuals affected by prostate cancer divided into low, intermediate and high risk based on the aggressiveness of the tumour, and in healthy individuals.

### DETAILED DISCLOSURE OF THE INVENTION

According to the present invention, the method for the in vitro/ex vivo diagnosis of prostate cancer in an individual comprises the following steps.

In a first step, a urine sample of the individual is provided. The urine sample is preferably obtained after performing prostate massage on the patient.

In a second step, the zinc in the urine sample is quantified. Preferably, the quantification is carried out by means of colorimetric test.

In a third step, the zinc value is compared with a reference value. The reference value corresponds to a value typical of an individual not affected by prostate cancer.

The method according to the present invention allows not only the diagnosis of prostate cancer but also allows evaluation of the aggressiveness thereof. The markers according to the present invention are differently expressed in the urines of patients with low, intermediate and high risk of progression of the disease. By “aggressiveness of the tumour” we mean the level of risk of progression of the disease.

Preferably, in the second step of the above-mentioned method also the total PSA (prostate specific antigen) (utPSA) is quantified and in the third step the quantified value of total PSA (utPSA) is compared with a reference value.

Even more preferably, in the second step the free PSA (prostate specific antigen) (ufPSA) is quantified and in the third step the quantified value of free PSA (ufPSA) is compared with a reference value.

Preferably the quantification of the total PSA (utPSA) and/or of the free PSA (ufPSA) is performed by means of immunoassay, spectrometric method, spectrophotometric method, colorimetric method, electrophoretic method, complexometric method, amperometric method, even more preferably immunoassay. The PSA sequence is provided as SEQ ID NO:1.

Even more preferably, in the second step a further prostate cancer biomarker is quantified and in the third step the quantified value of said biomarker is compared with a reference value. Said biomarker is selected from the group consisting of prostatic acid phosphatase (PAP), fibrinolysin, profibrinolysin, citrate, fructose, putrescine, spermidine, spermine, spermine oxidase, prostaglandins, calcium, copper.

Said biomarker is preferably spermine (C₁₀H₂₆N₄) or spermine oxidase (SEQ ID NO:2). Even more preferably, it is spermine oxidase. Preferably quantification of the spermine or the spermine oxidase is performed by means of immunoassay, spectrometric method, spectrophotometric method, colorimetric method, electrophoretic method, complexometric method, amperometric method, even more preferably immunoassay.

Preferably, in the method according to the invention the values of the biomarkers (zinc, total PSA (utPSA), free PSA (ufPSA), spermine, spermine oxidase etc.) are normalized with respect to the value of the creatinine so as to increase the accuracy of the test (as will be explained in further detail in the example).

In this preferred embodiment, therefore, the method according to the invention comprises the following steps.

In a first step, a urine sample of the individual is provided.

In a second step, the zinc in the urine sample is quantified.

In a third step, a molecule having molecular formula C₄H₇N₃O (creatinine) in the urine sample is quantified. Preferably the quantification is carried out by means of immunoassay, enzymatic method or Jaffe method.

In a fourth step, the quantified value of zinc is normalized with the quantified value of a molecule having molecular formula C₄H₇N₃O (creatinine).

In a fifth step, the zinc normalized value is compared with a reference value. The reference value corresponds to a typical value of an individual not affected by prostate cancer.

Also in this embodiment, the total PSA (prostate specific antigen) value (utPSA) is preferably quantified and normalized and even more preferably the free PSA (prostate specific antigen) value (ufPSA).

Preferably, therefore, in the second step also the total PSA (prostate specific antigen) (utPSA) is quantified, in the fourth step the quantified value of total PSA (utPSA) is normalized with the quantified value of a molecule having molecular formula C₄H₇N₃O (creatinine), and in the fifth step the normalized value of total PSA (utPSA) is compared with a reference value.

Even more preferably, in the second step also the free PSA (prostate specific antigen) (ufPSA) is quantified, in the fourth step the quantified value of free PSA (ufPSA) is normalized with the quantified value of a molecule having molecular formula C₄H₇N₃O (creatinine), and in the fifth step the normalized value of free PSA (ufPSA) is compared with a reference value.

Even more preferably, in the second step a further prostate cancer biomarker is quantified, in the fourth step the quantified value of said biomarker is normalized with the quantified value of a molecule having molecular formula C₄H₇N₃O (creatinine), and in the fifth step the normalized value of said biomarker is compared with a reference value. Said biomarker is selected from the group consisting of prostatic acid phosphatase (PAP), fibrinolysin, profibrinolysin, citrate, fructose, putrescine, spermidine, spermine, prostaglandins, calcium, copper. Said biomarker is preferably spermine or spermine oxidase.

The kit for use in the method according to the present invention comprises
means for collecting and preparing a urine sample from an individual;
means for quantifying the zinc.

Preferably, the kit also comprises:
means for quantifying the total PSA (utPSA); and/or
means for quantifying the free PSA (ufPSA); and/or
means for quantifying other prostate cancer biomarkers, preferably spermine or spermine oxidase; and/or
means for quantifying a molecule having molecular formula C₄H₇N₃O (creatinine).

### EXAMPLE

Urine samples were collected from 110 patients aged between 51 and 86 with prostate biopsy indication.

The urine samples were collected after digital rectal examination and prostatic massage consisting of 3 digital compressions in each lobe starting from the base, towards the median part and the apex for a duration of 180 seconds. The samples were stirred and frozen in Falcon tubes at −80° C. within 30 minutes from collection.

12 bioptic samples were also taken from each patient, used for the normal diagnostic procedure by the anatomical pathology department. The histological evaluation identified the presence of neoplastic cells in 53 patients (PCa). Of the remaining patients, 29 were free from disease, in 16 a benign pathological situation was found, and in 12 a potentially pre-cancerous situation. This indicates that approximately 55% of the patients, suspect for PSA/DRE/ECO, underwent superfluous biopsy.

The urine samples were thawed and the presence of utPSA and ufPSA biomarkers was evaluated by means of ELISA assay (Sigma Aldrich), the zinc was quantified by means of colorimetric test (Zinc assay test, Sigma Aldrich), the spermine was quantified by means of ELISA assay (MyBiosource), and the spermine oxidase was quantified by means of ELISA assay (MyBiosource).

Since the quantity of the biomarkers in the urine depends on the volume of urine collected and on its concentration, a calculation was carried out called “normalization to creatinine” to increase the accuracy of the test. Creatinine is a substance produced by the body, it is excreted via the urine and its level is an indicator of the urine concentration. The higher the creatinine value, the greater the urine concentration. The lower the creatinine value, the greater the urine dilution.

To eliminate possible confusing factors, cases with pre-cancerous situations and benign pathologies were excluded.

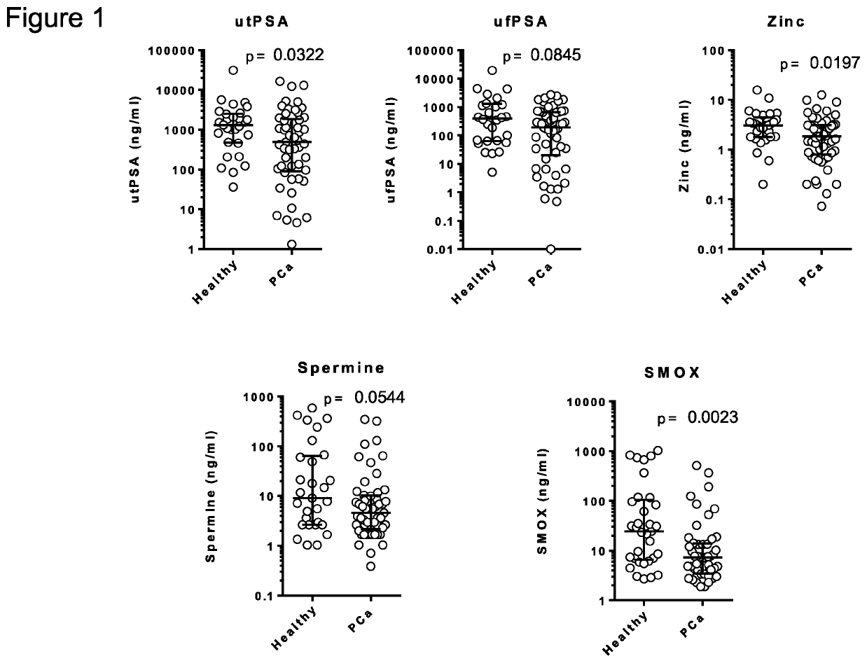

From the absolute values of the biomarkers it was possible to identify a statistically significant difference between healthy and ill patients (see FIG. 1).

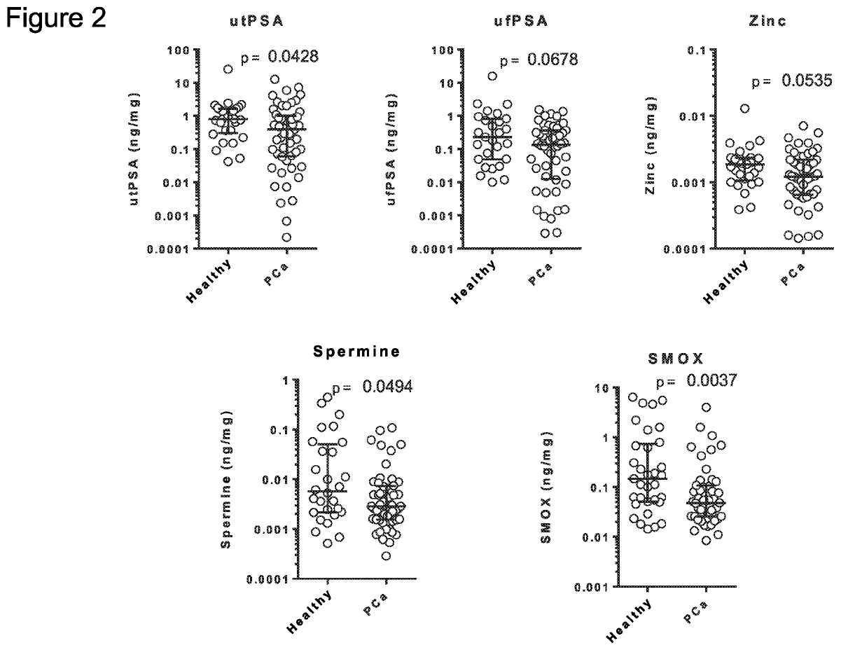

After normalization to creatinine an even more significant difference was observed between ill and healthy patients for all 5 of the markers studied (see FIG. 2).

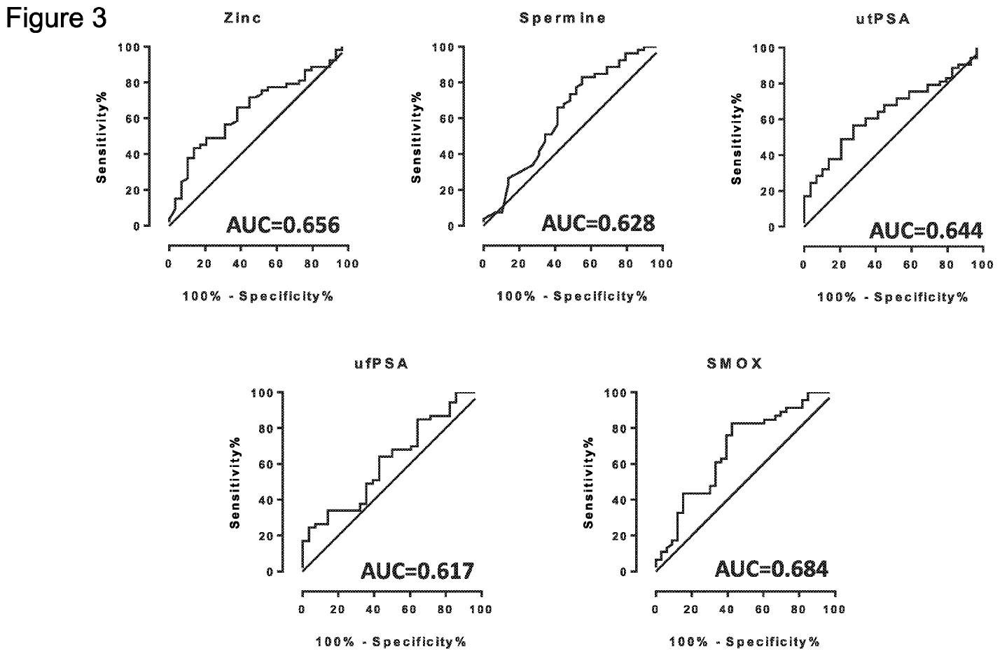

The values of Zn, Spermine, utPSA, ufPSA, spermine oxidase correlate with the presence of tumour, with an AUC of 0.656, 0.628, 0.644, 0.617, 0.684 respectively (see FIG. 3).

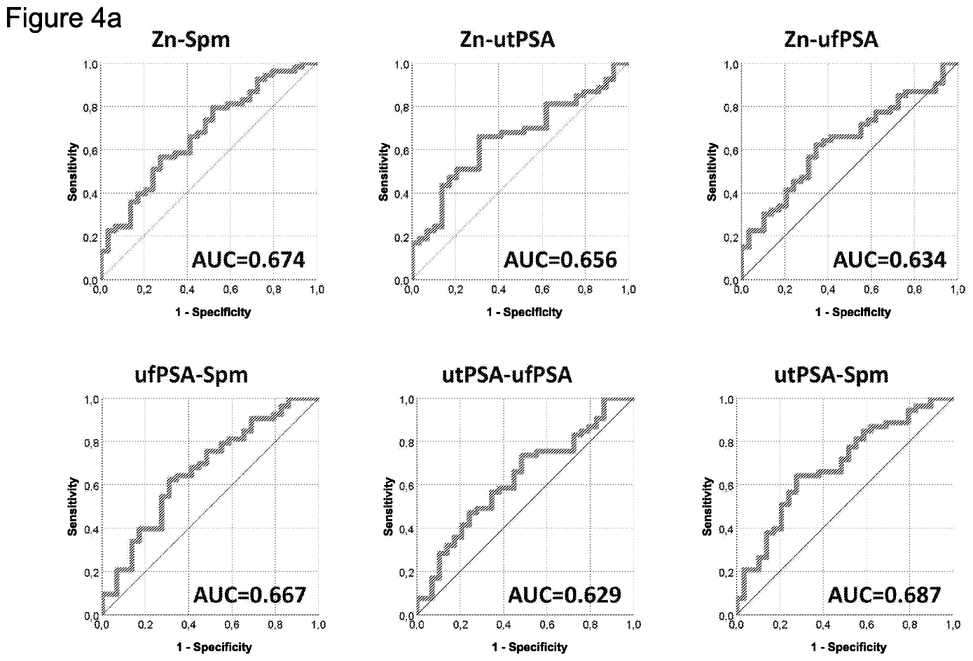

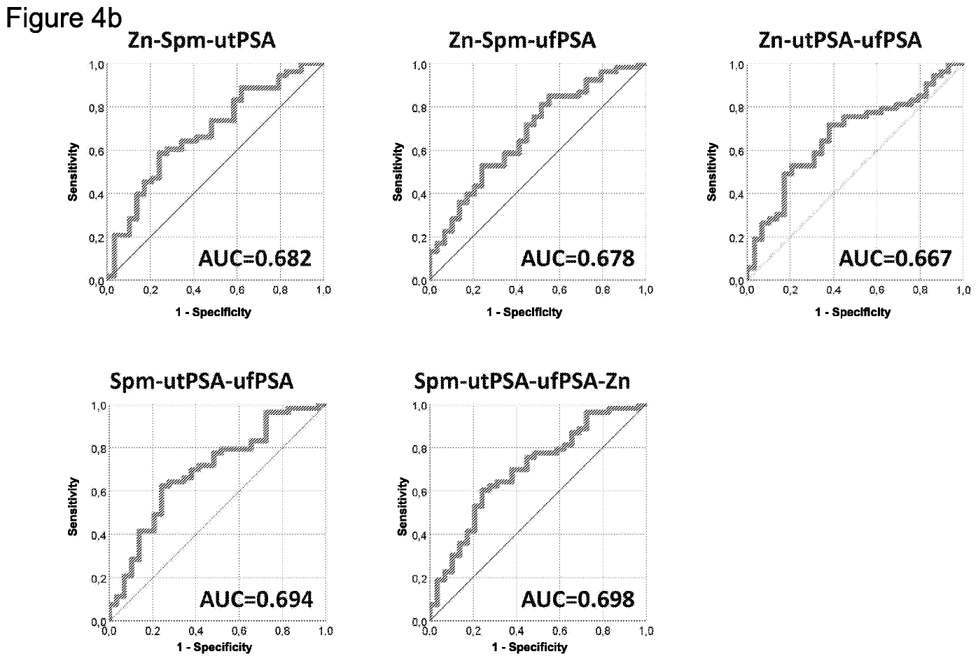

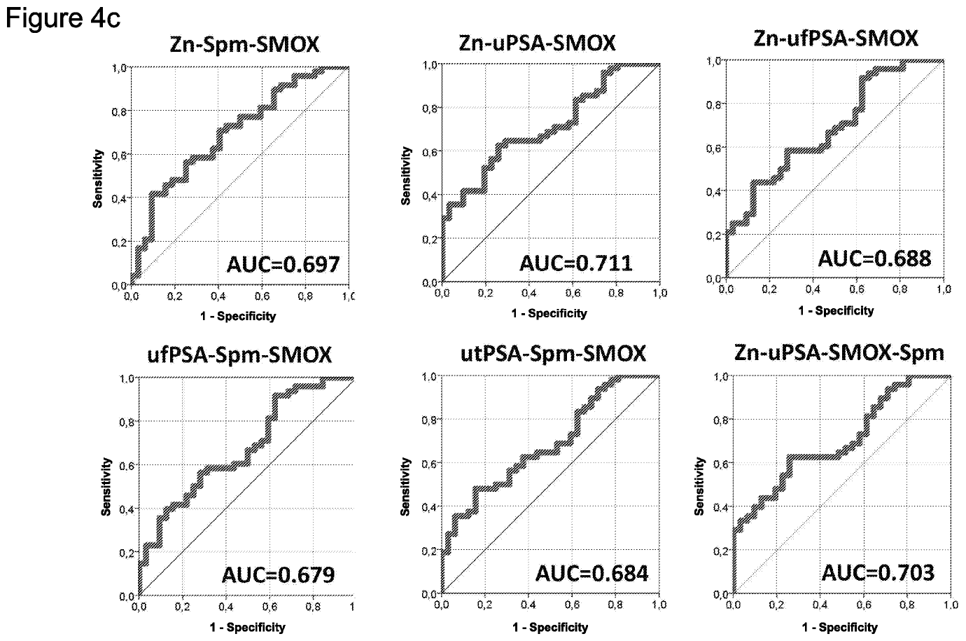

By carrying out a multivariate analysis it is observed that the combination of several factors increases the power of the test in correlating the level of biomarkers and prostate tumour (see FIGS. 4a, 4b and 4c).

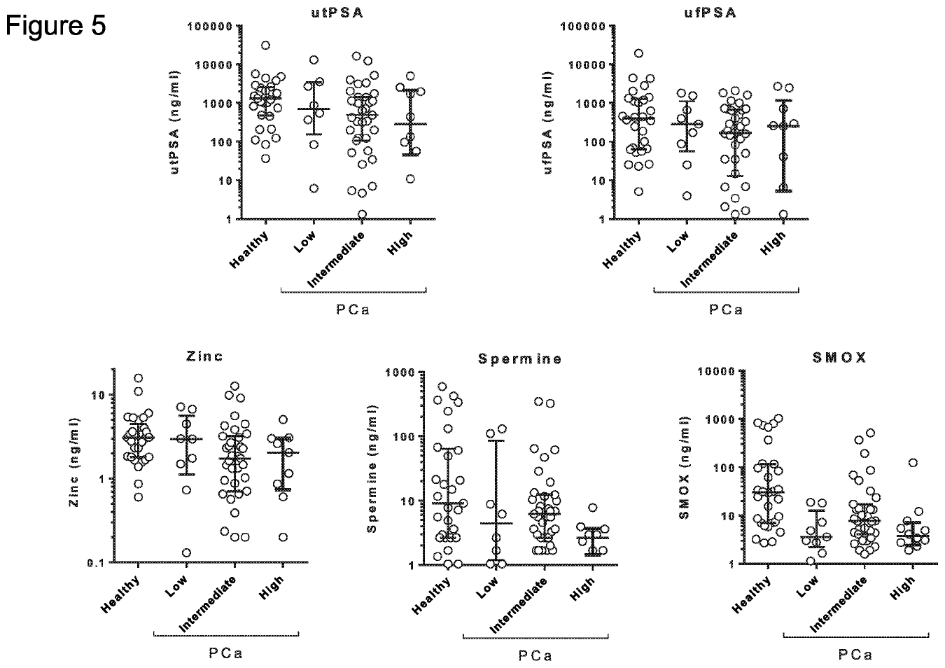

Analysing the same values after dividing the patients based on the aggressiveness of the tumour (low, intermediate, high risk), it is highlighted that each of the five markers analysed decreases to a greater extent in the patients with tumour at a more advanced stage (see FIG. 5).

From the results of the example, the advantages of the present invention are evident.

Analysis in urine samples of the absolute values of the single biomarkers zinc, total PSA, free PSA, spermine, spermine oxidase is already indicative of prostate cancer. Analysis of the normalized values with respect to the creatinine allows even more accurate information to be obtained. Simultaneous analysis of several biomarkers allows even more reliable identification of the patients with prostate cancer. In particular, the combination Zn, utPSA and spermine oxidase allows improved discrimination between a healthy patient and a patient with prostate cancer (AUC=0.711).

For each of these analytes, the value with 80% sensitivity was identified as the optimal threshold.

The samples in which a biomarker is present in a quantity higher than the threshold value were considered positive.

In this way a scale from 0 to 3 (SCORE) was established, based on the number of positive biomarkers for each sample, and a probability of having prostate cancer (RISK) was defined.

TABLE 1

| SCORE | RISK |
|---|---|
| 0 | 0 |
| 1 | 14 |
| 2 | 43 |
| 3 | 66 |

As can be seen in Table 1, for individuals with no positive biomarker the probability of having prostate cancer is nil (0%).

The probability increases to 14%, 43%, 66% in individuals with 1, 2, 3 positive biomarkers respectively. These results indicate that, based on use of the biomarkers according to the invention, it would be possible to identify individuals with a very low probability of prostate cancer and therefore avoid them undergoing invasive clinical examinations.

The method according to the present invention represents a rapid inexpensive way of obtaining a reliable diagnosis, avoiding invasive procedures.

1. A method for the in vitro/ex vivo diagnosis of a prostate cancer and/or of the aggressiveness thereof in an individual comprising the steps of:
a) providing a urine sample of the individual;
b) quantifying the zinc in the urine sample;
c) comparing the quantified value of zinc with a reference value.

2. The method according to claim 1, wherein in step b) the total PSA (prostate specific antigen) (utPSA) and/or the free PSA (prostate specific antigen) (ufPSA) are quantified and in step c) the quantified value of total PSA (utPSA) is compared with a reference value and/or the quantified value of free PSA (ufPSA) is compared with a reference value.

3. The method according to claim 1, wherein in step b) the spermine is also quantified and in step c) the quantified value of spermine is compared with a reference value.

4. The method according to claim 1, wherein in step b) the spermine oxidase (SMOX) is also quantified and in step c) the quantified value of spermine oxidase is compared with a reference value.

5. The method according to claim 4, wherein in step b) the total PSA (prostate specific antigen) (utPSA), the zinc and the spermine oxidase (SMOX) are quantified and in step c) the quantified values of total PSA (prostate specific antigen) (utPSA), zinc and spermine oxidase (SMOX) are compared with a reference value.

6. A method for the in vitro/ex vivo diagnosis of a prostate cancer and/or the aggressiveness thereof in an individual comprising the steps of:
a′) providing a urine sample of the individual;
b′) quantifying the zinc in the urine sample;
c′) quantifying in the urine sample a molecule having molecular formula C₄H₇N₃O (creatinine);
d′) normalizing the quantified value of zinc with the quantified value of a molecule having molecular formula C₄H₇N₃O (creatinine);
e′) comparing the normalized value of zinc with a reference value.

7. The method according to claim 6, wherein in step b′) the total PSA (prostate specific antigen) (utPSA) and/or the free PSA (prostate specific antigen) (ufPSA) are quantified, in step d′) the quantified value of total PSA (utPSA) is normalized with the quantified value of a molecule having molecular formula C₄H₇N₃O (creatinine) and/or the quantified value of free PSA (ufPSA) is normalized with the quantified value of a molecule having molecular formula C₄H₇N₃O (creatinine), and in step e′) the normalized value of total PSA (utPSA) is compared with a reference value and/or the normalized value of free PSA (ufPSA) is compared with a reference value.

8. The method according to claim 6, wherein in step b′) the spermine is quantified, in step d′) the quantified value of spermine is normalized with the quantified value of a molecule having molecular formula C₄H₇N₃O (creatinine), and in step e′) the normalized value of spermine is compared with a reference value.

9. The method according to claim 6, wherein in step b′) the spermine oxidase (SMOX) is quantified, in step d′) the quantified value of spermine oxidase (SMOX) is normalized with the quantified value of a molecule having molecular formula C₄H₇N₃O (creatinine), and in step e′) the normalized value of spermine oxidase (SMOX) is compared with a reference value.

10. The method according to claim 9, wherein in step b′) the total PSA (prostate specific antigen) (utPSA), the zinc and the spermine oxidase (SMOX) are quantified, in step d′) the quantified values of total PSA (prostate specific antigen) (utPSA), zinc and spermine oxidase (SMOX) are normalized with the quantified value of a molecule having molecular formula C₄H₇N₃O (creatinine), and in step e′) the normalized values of total PSA (prostate specific antigen) (utPSA), zinc and spermine oxidase (SMOX) are compared with a reference value.

11. The method according to claim 1, wherein the quantification of the zinc is performed by means of colorimetric test.

12. The method according to claim 2, wherein the quantification of the total PSA (utPSA) and/or of the free PSA (ufPSA) is performed by means of immunoassay, spectrometric method, spectrophotometric method, colorimetric method, electrophoretic method, complexometric method, amperometric method.

13. A kit for use in the method according to claim 1 comprising:
means for collecting and preparing a urine sample from an individual;
means for quantifying the zinc.

14. The kit for use in the method according to claim 10, further comprising
means for quantifying the total PSA (utPSA);
means for quantifying the spermine oxidase (SMOX); and
means for quantifying a molecule having molecular formula C₄H₇N₃O (creatinine).

15. The method according to claim 7, wherein the quantification of the total PSA (utPSA) and/or of the free PSA (ufPSA) is performed by means of immunoassay, spectrometric method, spectrophotometric method, colorimetric method, electrophoretic method, complexometric method, amperometric method.
